(19) United States
(12) Patent Application Publication
(10) Pub. No.: US 2011/0082435 A1
(43) Pub. Date: Apr. 7, 2011

(54) DIAPER WITH REMOVEABLE WIPE POUCH

(76) Inventor: Tensele Davis, Lorain, OH (US)

(21) Appl. No.: 12/571,955

(22) Filed: Oct. 1, 2009

(51) Int. Cl. A61F 13/15

(52) U.S. Cl. 604/385.06

(57) ABSTRACT

An improved diaper having an inner absorbent layer, and an outer waterproof layer, also has a powder layer, wherein the powder layer is located on the inner absorbent layer; and a release layer, wherein the release layer covers the powder layer, and is removeably attached to the diaper, such that the release layer keeps the powder layer in place when the diaper is not in use, and such that the release layer is removed before diaper use, thereby exposing the powder layer. A further improvement is a removeable moisture retaining pouch, wherein the pouch is of a size and shape to hold at least one diaper wipe, and wherein the pouch is removeably attached to the diaper outer waterproof layer.

### BACKGROUND

The present invention is in the field of diapers.

In the busy world of today, where parents are on the go with their babies all the time, it is easy to forget simple items, such as baby powder, or diaper wipes.

There are diapers in the prior art which have added talc or baby powder. However, the nature of the powder is to be loose, and therefore much of this powder may be lost before the diaper ever touches the baby's skin.

Further, there are diapers with pouches on the outside for holding wipes and or other items. However, once these diapers are on the baby, the pouch is no longer useful.

Therefore, it is desired to have an improved diaper wherein there is added talc or other powder for prevention of diaper rash, and the further comfort of the child, and wherein the powder is contained before use, such that all of the powder is present when the diaper goes on the baby. It is further desired to have a diaper wherein there is an attached wipe, and wherein the wipe is attached in a pouch which is removable, such that the wipe can be saved if it is not used, and which is resealable, such that a wipe could be removed and replaced, and the pouch would seal in the wipes moisture for further use.

### SUMMARY

An improved diaper having an inner absorbent layer, and an outer waterproof layer, also has a powder layer, wherein the powder layer is located on the inner absorbent layer; and a release layer, wherein the release layer covers the powder layer, and is removeably attached to the diaper, such that the release layer keeps the powder layer in place when the diaper is not in use, and such that the release layer is removed before diaper use, thereby exposing the powder layer. A further improvement is a removeable moisture retaining pouch, wherein the pouch is of a size and shape to hold at least one diaper wipe, and wherein the pouch is removeably attached to the diaper outer waterproof layer.

### BRIEF DESCRIPTION OF DRAWINGS

FIG. 1 is a top view of an open diaper according to an aspect of the invention.

FIG. 2 is a top view of an open diaper according to an aspect of the invention.

FIG. 3 is across sectional view from FIG. 2.

FIG. 4 is a back view of an open diaper according to an aspect of the invention.

FIG. 5 is a top view of a resealable pouch according to an aspect of the invention.

### DETAILED DESCRIPTION

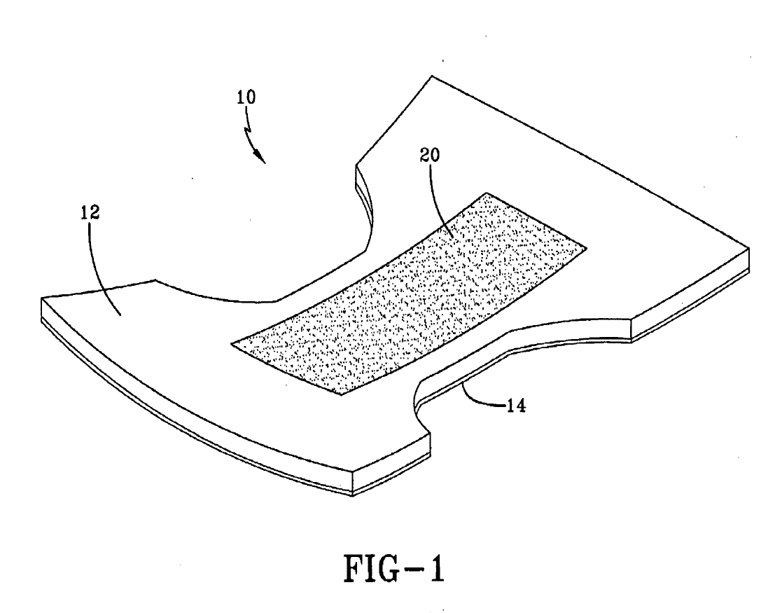

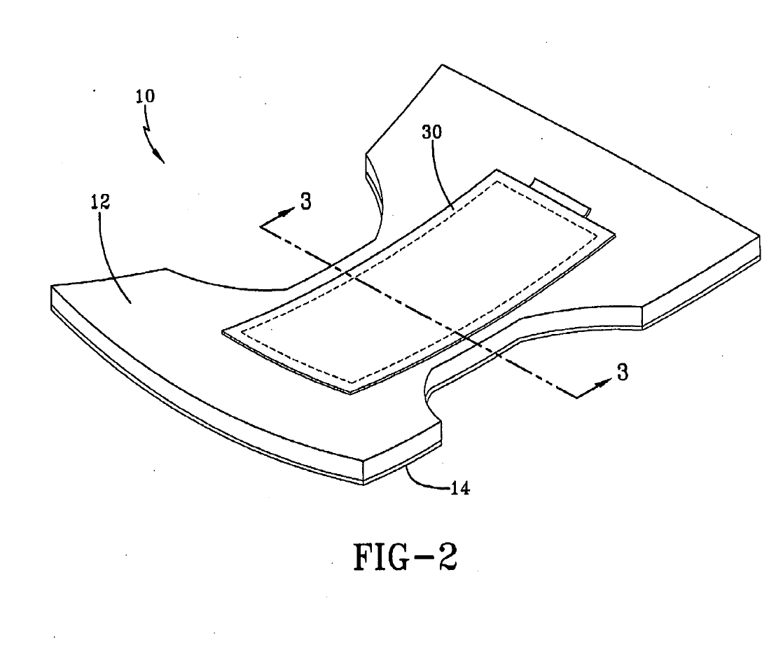

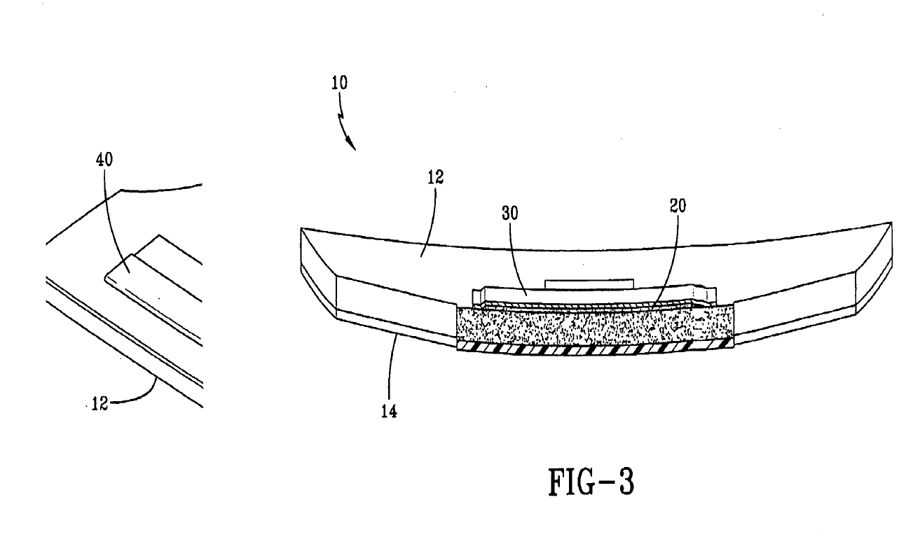

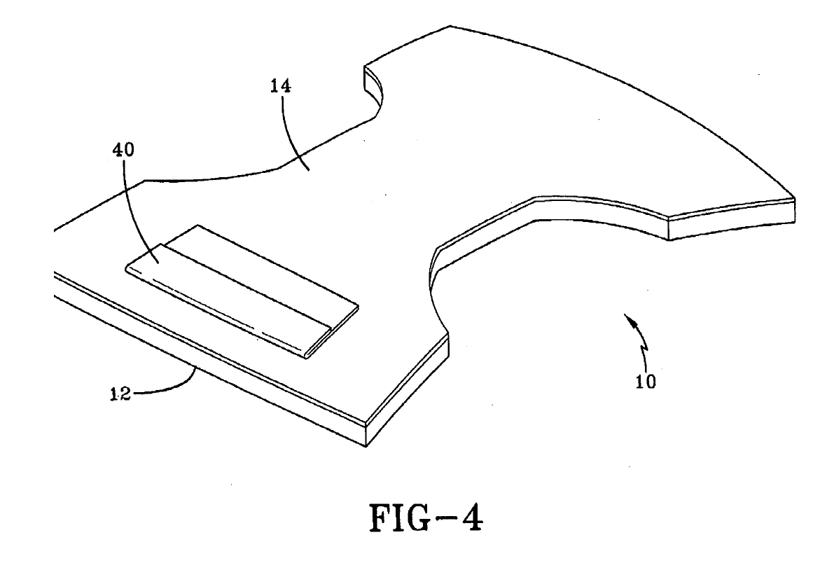

Various aspects of the invention are presented in FIGS. 1-6 which are not drawn to scale and in which like components are numbered alike. A diaper 10 is shown, having an inner absorbent layer 12, and an outer waterproof layer 14. Referring now to FIGS. 1-3, according to an aspect of the invention, the improvement comprises a powder layer 20, wherein the powder layer 20 is located on the inner absorbent layer 12, and, a release layer 30. The release layer 30 covers the powder layer 20, and is removeably attached to the diaper 10, such that the release layer 30 keeps the powder layer 20 in place when the diaper 10 is not in use. The release layer 30 is removed before diaper 10 use, thereby exposing the powder layer 20.

According to an aspect of the invention, the powder layer 20 is a fluid absorbing powder, such as a baby powder, corn starch, or talc powder, although any appropriate powder is deemed to be within the purview of this invention. The powder layer is placed on the inner absorbent layer 12 such that when the release layer 30 is removed, the powder layer 20 will be against the baby's bottom and genitals to soothe irritation, and help prevent diaper rash.

According to a further aspect of the invention, the diaper improvement comprises a removeable moisture retaining pouch 40, and at least one diaper wipe 50 contained within the removeable pouch 40. The pouch 40 is of a size and shape to hold at least one diaper wipe 50, and the pouch 40 is removeably attached to the diaper outer waterproof layer 14.

According to an aspect of the invention the removeable pouch 40 is removeably attached with adhesive. The removeable pouch 40 may also be removeably attached with Velcro. These are just examples of methods for removeably attaching the pouch, any appropriate method is deemed to be within the purview of this invention.

According to a further aspect of the invention, the removeable pouch 40 is resealable, such that the wipe 50 may be removed and replaced, and the pouch 40 may then be resealed such as to allow the diaper wipe 50 to continue to retain moisture. The removeable pouch may be resealable by any known appropriate means 60, such as adhesive or Velcro.

This invention thus allows the diaper alone to be thrown into the diaper bag, and the caregiver has everything they need. The powder is in place to prevent diaper rash, and the wipe is attached for use when changing. Because the pouch 40 is resealable, more than one wipe could be placed in the pouch 40, and thus if one is used, any remaining wipes could be saved in the pouch for later use.

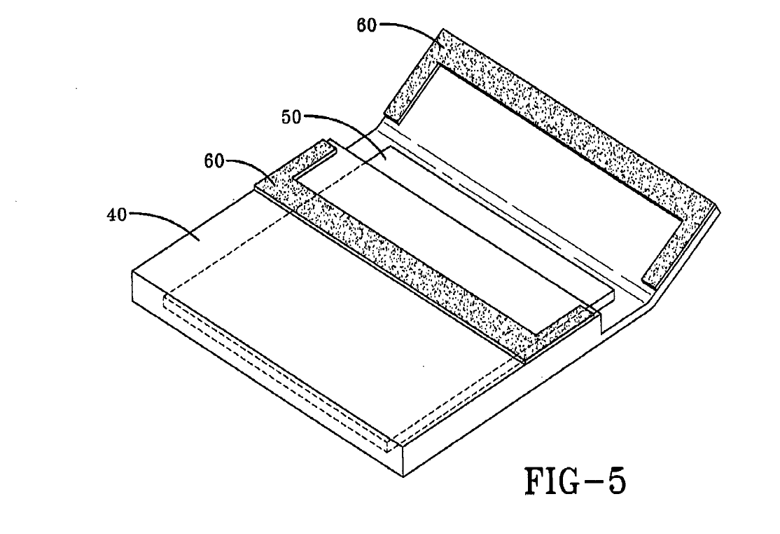

According to an aspect of the invention, FIG. 5 shows the pouch 40 open, with a wipe 50 showing. The reasealable means 60 is located on the open flap, and the pouch body, such that when the flap is folded down, the wipe is enclosed. There are many possible configurations for sealing the pouch, and this example is not intended to limit such possibilities in any way.

The improvements are talked about separately, but an improved diaper could have either the powder layer under the release layer or the removeable pouch, or it could have both features.

I claim:

1. An improved diaper having an inner absorbent layer, and an outer waterproof layer, the improvement comprising:
a powder layer, wherein said powder layer is located on said inner absorbent layer; and,
a release layer, wherein said release layer covers said powder layer, and is removeably attached to the diaper, such that said release layer keeps said powder layer in place when the diaper is not in use, and such that said release layer is removed before diaper use, thereby exposing said powder layer.

2. The improved diaper of claim 1 wherein said powder is a fluid absorbing powder.

3. The improved diaper of claim 1 wherein said powder is corn starch.

4. The improved diaper of claim 1 wherein said powder is baby powder.

5. The improved diaper of claim 1 wherein said powder is talc powder.

6. An improved diaper having an inner absorbent layer, and an outer waterproof layer, the improvement comprising:
a removeable moisture retaining pouch, wherein said pouch is of a size and shape to hold at least one diaper wipe, wherein said pouch is removeably attached to the diaper outer waterproof layer.

7. The improved diaper of claim 6 further comprising;
at least one diaper wipe contained within said removeable pouch.

8. The improved diaper of claim 6 wherein said removeable pouch is removeably attached with adhesive.

9. The improved diaper of claim 6 wherein said removeable pouch is removeably attached with Velcro.

10. The improved diaper of claim 6 wherein said pouch is resealable, such that said wipe may be removed and replaced, and said pouch may then be resealed such as to allow said diaper wipe to continue to retain moisture.

11. The improved diaper of claim 6 wherein said pouch is resealable by adhesive.

12. The improved diaper of claim 6 wherein said pouch is resealable by Velcro.
